(19) United States
(12) Patent Application Publication
(10) Pub. No.: US 2026/0363982 A1
(43) Pub. Date: Nov. 19, 2026

(54) A METHOD OF PROVIDING HIGH SPF TO A TOPICAL SURFACE OF A BODY

(71) Applicant: Conopco, Inc., d/b/a UNILEVER, Hoboken, NJ (US)

(72) Inventors: Aditi Jayavant KULKARNI, Mumbai (IN); Praful Gulab Rao LAHORKAR, Bangalore (IN); Kalavathi RAMANAN, Chennai (IN); Ashish Anant VAIDYA, Bangalore (IN); Jonish VARSHNEY, New Delhi (IN)

(73) Assignee: Conopco, Inc., d/b/a UNILEVER, Hoboken, NJ (US)

(21) Appl. No.: 19/118,865

(22) PCT Filed: Oct. 16, 2023
(86) PCT No.: PCT/EP2023/078633

§ 371 Date: Apr. 7, 2025

(30) Foreign Application Priority Data
Oct. 20, 2022 (EP) 22202811.0

(51) Int. Cl. A61K 8/49; A61K 8/36; A61K 8/37; A61K 8/44; A61Q 17/04

(52) U.S. Cl. CPC A61K 8/4946; A61K 8/36; A61K 8/37; A61K 8/44; A61Q 17/04; A61K 2800/30; A61K 2800/591; A61K 2800/87

(57) ABSTRACT

The present invention relates to a method of delivering high Sun Protection Factor (SPF) and UVA protection factor (UVAPF) to a topical surface of a human body. The method provides for synergistic benefit of SPF and UVA PF over and above that which can be delivered through each of two individual steps carried out sequentially viz. a washing off step followed by the step of applying a leave on composition on to the same skin surface. The method comprises washing the skin with a foam composition generated from a container comprising a pump, the foam composition comprising a water soluble sunscreen followed by applying a leave-on composition which could comprise any sunscreen.

### FIELD OF THE INVENTION

The present invention relates to a method of delivering high Sun Protection Factor (SPF) to a topical surface of a body preferably the human body. The method more preferably provides for synergistic benefit of SPF over and above that which can be delivered through each of two individual steps carried out sequentially viz. washing of the skin with a specific cleansing foam composition delivered from a container with a pump followed by the step of applying a sunscreen composition on to the same skin surface.

### BACKGROUND OF THE INVENTION

Solar radiation includes about 5% ultraviolet (UV) radiation, wavelength of which is between 200 nm and 400 nm. It is further classified into three regions: from 320 to 400 nm (UV-A), 290 to 320 nm (UV-B) and from 200 to 290 nm (UV-C). Exposure to UV-A and UV-B radiation for short period is known to cause reddening of the skin and localized irritation. Continued and prolonged exposure can lead to sunburn, melanoma and formation of wrinkles. It is also reported that UV radiation causes significant damage to hair. Therefore, people desire to protect their skin and hair from the harmful effects of both UV-A and UV-B radiation.

Various cosmetic preparations that one can apply on to the skin such as creams, lotions or gels have been reported for preventing and/or protecting the skin from harmful effects of ultraviolet radiation. These cosmetic compositions usually comprise different types of organic sunscreen agents, especially ones capable of absorbing the UV-A and/or UV-B radiation present in the sun's rays. Thus, both UV-A and UV-B sunscreens are usually incorporated in photo-protective compositions so as to provide protection over the entire range of UV radiation. Such leave-on compositions are applied on to skin before a person goes outdoors and they remain thereon till the person has a next wash.

An alternate method of application of sunscreens on the skin is through wash-off products like soaps, face wash or body wash products where the sunscreens are incorporated in the skin cleansing products and are delivered on to the skin concurrent with the washing process. However, it is an extremely difficult challenge to deliver sunscreens on to skin when the primary purpose of wash-off products is to remove the dirt and oils from the skin surface and together with that, the actives in the wash-off products are also highly likely to get washed away. Thus, enhanced deposition of actives through wash-off products is an on-going challenge.

The present inventors with their years of experience in understanding the nature of available sunscreens; the knowledge of interfacial science of surfactant systems and through extensive experimentation set out find a solution to the problem of delivering high SPF on to skin. They hit upon a unique two step method that provides for synergistic improvement in SPF as compared to each of the steps individually. The method comprises the step of washing the skin with a foam composition comprising water soluble sunscreens which is generated from a container fitted with a foam generating pump; followed by the step of applying a leave-on sunscreen composition on to skin.

EP1261688 (Unilever, 2001) discloses a personal wash composition which deposits high levels of sunscreen (SPF>2) while maintaining good lather (i.e. suffer minimal lather degradation over time relative to compositions with more “oily” sunscreens). Enhanced deposition is found from both bar and liquid compositions and is based on the solubility or non-solubility of the sunscreen used.

The present inventors have found that there is a limit to the SPF that can be achieved with either a wash-off sunscreen composition or a leave-on sunscreen composition, the latter being more efficacious. Incorporation of higher amount of sunscreens as taught in the prior art does not, in general, provide for continuous increase+ in the deposition efficacy and thereby the SPF achieved.

It is therefore an object of the present invention to obviate at least some drawbacks of the prior art and provide a method which delivers enhanced and even synergistic SPF as compared to the known art.

It is another object of the present invention to provide for a method that provides SPF of higher than 10 and a UVAPF (UV-A protection factor) of higher than 4.

### SUMMARY OF THE INVENTION

According to the first aspect of the present invention, there is provided a method of providing high SPF to a topical surface of a body comprising the steps of
(a) washing the surface with a wash-off composition generated as a foam from a container comprising a pump, said wash-off composition comprising
(i) 0.1 to 10 wt % of a water-soluble UVA sunscreen;
(ii) 0.1 to 10 wt % of a water-soluble UVB sunscreen;
(iii) 3 to 80 wt %, preferably 6 to 80 wt % a mild surfactant selected from one or more of an anionic, zwitterionic and non-ionic surfactant; and
(iv) a cosmetically acceptable carrier;
preferably diluted with water followed by
(b) rinsing said surface with water; followed by
(c) applying a leave-on composition comprising a sunscreen and a cosmetically acceptable vehicle, on said surface.

The second aspect of the present invention relates to a kit comprising
(a) a hand held container comprising a pump capable of generating a foam, said container comprising a wash-off composition comprising 0.1 to 10 wt % of a water-soluble UVA sunscreen; 0.1 to 10 wt % of a water-soluble UVB sunscreen; 3 to 80 wt %, preferably 6 to 80 wt % a mild surfactant selected from one or more of an anionic, zwitterionic or non-ionic surfactant; and a cosmetically acceptable carrier;
(b) a leave-on composition comprising a sunscreen and a cosmetically acceptable vehicle; and
(c) Instructions for use.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The disclosure of the invention as found herein is to be considered to cover all embodiments as found in the claims as being multiply dependent upon each other irrespective of the fact that claims may be found without multiple dependency or redundancy. Where a feature is disclosed with respect to a particular aspect of the invention (for example a method of the invention), such disclosure is also to be considered to apply to any other aspect of the invention (for example a kit of the invention) mutatis mutandis. Preferably, the method or the use is non-therapeutic or cosmetic in nature.

By “a wash-off composition” as used herein, is meant to include a composition for cleaning topical surfaces of mammals, especially humans. This composition is particularly useful for use on the sun-exposed parts of the body. Such a composition is generally of the rinse off type which means that the composition is high in surfactants which are known to help in cleaning surfaces to make them free of oils and dirt. The wash-off composition is generally used by diluting with water as it is applied on to skin, scalp or hair, after which the consumer works up a lather to ensure that the dirt and oil on the surface are solubilized in the micelles of the surfactant solution and the body is then rinsed with copious amounts of water to ensure that the surface is substantially free of the composition. The wash-off composition of the present invention is included in a container that is fitted with a pump. When the pump on the container is actuated, the composition issues as a foam from the container which is then used by the consumer for washing the desired surface of the body. “Skin” as used herein is meant to include skin on the face and body (e.g., neck, chest, back, arms, underarms, hands, legs, buttocks and scalp) and especially to the sun exposed parts thereof. The wash-off composition of the invention is also of relevance to application on any other keratinous substrates of the human body other than skin e.g. scalp and hair where products may be formulated with specific aim of providing photo-protection. It has been observed that with the use of the wash-off composition of the method of the present invention, the dirt and oil on the skin surface are substantially removed during the washing and rinsing process. Further, surprisingly, the specific combination of the sunscreens incorporated ensures that they deposit in much higher amounts than is generally expected from rinse off products, in such a way that the deposited sunscreens interact synergistically with the sunscreens applied thereafter from the leave-on composition to provide the enhanced SPF and UVAPF benefit. The enhanced deposition leads to delivering high Sun Protection Factor (SPF). By high SPF as per this invention is meant a composition that preferably has an SPF at least 10, more preferably at least 15, more preferably higher than 20, further more preferably higher than 30, yet more preferably higher than 50, in many cases higher than 80, and in optimum cases higher than 100. Most preferably the invention provides for SPF in the range of 20 to 50. The SPF could be as high as 160. The SPF in the present invention is measured using transmittance measurement technique. The method of the invention also delivers high UVAPF. By high UVAPF is meant that the method delivers UVAPF (initial) of at least 4, preferably higher than 8, further preferably higher than 12, further more preferably higher than 16, and most preferably higher than 20. In especially preferred aspects, the present invention delivers UVAPF value in the range of 4 to 16. The UVAPF could be as high as 40.

The wash-off composition which is used in the first step of the method of the invention comprises 0.1 to 10 wt % of a water-soluble UVA sunscreen; 0.1 to 10 wt % of a water-soluble UVB sunscreen; 3 to 80 wt %, preferably 6 to 80 wt % a mild surfactant selected from one or more of an anionic, zwitterionic and non-ionic surfactant; and a cosmetically acceptable carrier.

By water soluble sunscreen, whether of the UVA type, or of the UVB type, is meant that the solubility in water of the sunscreen is higher than 10 g/l preferably higher than 50 g/l at 25 °C. The wash-off composition is included in a container. The container is preferably capable of being hand-held. The container comprises a pump at its head such that on actuation of the pump by pressing down with a finger or a thumb the composition mixes with air and issues from the container as a foam. The various ways in which the container with the pump could be delivered are described below.

The container comprises a sprayable foamable composition. It may be delivered using a spray device that contains the foamable composition. The spray device may comprise a bottle having a nozzle and a spray pump dispenser. The invention thus provides a cleaning system comprising a spraying device. The spraying device comprises a container holding the foamable wash-off composition, a spray head, and a liquid supply arrangement for transferring the foamable wash-off composition from the container to the spray head to form the foam. The foam generated generally has a density of less than 0.4 g/ml when ejected from the spray device through the spray head.

The spraying device used in the present invention preferably comprises a container having an internal volume of 25 to 1000 ml, more preferably of 50 to 800 ml, even more preferably 60 to 750 ml and most preferably of 75 to 500 ml. The spraying device preferably comprises a positive displacement pump that acts directly on the foamable wash-off composition. The pump draws the composition up into the liquid supply arrangement and transfers the composition to the spray head, from which it is discharged in the form of a foam, preferably through a nozzle.

In the spraying device the dispensing of the wash-off composition is preferably powered by a user's efforts, i.e, the liquid cleaning composition is not dispensed under pressure by simply actuating a valve and requires manual triggering. The spraying device employed in accordance with the present invention is preferably selected from a trigger spray foam bottle, a squeeze foam bottle and a foam pump. Most preferably the spraying device is a squeeze foam bottle or a foam pump. In another preferred embodiment, the spray device is configured to mix the liquid cleaning composition with air before it is dispensed from the spray head. The composition can be placed into the reservoir of a plastic squeeze bottle which contains a foaming spray head or other foam producing means. Squeezing the container causes the wash-off composition to leave the reservoir and enter an air-mixing or foaming chamber via an internal dip tube. The foam produced in the foaming chamber is often passed through a homogenizing element interposed between the air-mixing chamber and the discharge orifice to homogenise and control the consistency of the discharged foam. Further compression of the foam discharges the foam from a discharge cap as a uniform non-pressurised aerated foam. Alternatively the side walls of the container may be rigid and the dip tube may be fitted with a pump that is actuated by a push button. When composition is drawn by the pump through to the air mixing or foaming chamber, the desired foam is produced.

The preferred UVB sunscreen for use in the wash-off composition is phenyl benzimidazole sulphonic acid (PBSA) or benzylidene camphor sulfonic acid, preferably PBSA.

PBSA has the chemical structure:

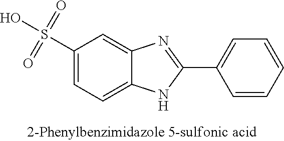 [SMILES: O=S(=O)(O)c1ccc2[nH]c(-c3ccccc3)nc2c1]

PBSA also known as Ensulizole is commercially available as Eusolex 232 (from Merck KGaA). PBSA is also available under the brand names Neo Heliopan Hydro (from Symrise), Parsol HS (from DSM) and Sunsafe ES (from Uniproma). It is generally used in the water solubilized form after neutralization to deliver desired benefit.

Benzylidene camphor sulfonic acid has the chemical structure: It is generally used in the water solubilized form pre/post neutralization to deliver desired benefit.

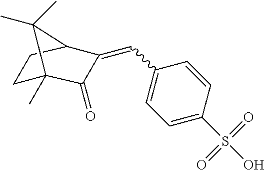 [SMILES: CC12CCC(C(=Cc3ccc(S(=O)(=O)O)cc3)C1=O)C2(C)C]

The preferred UVA sunscreen for use in the wash-off composition is one or more of disodium phenyl dibenzimidazole tetrasulfonate (Neoheliopan AP) or terephthalylidene dicamphor sulfonic acid (TDSA).

Di sodium phenyl dibenzimidazole tetra sulfonate also known as bisdisulizole disodium has the chemical structure as given below:

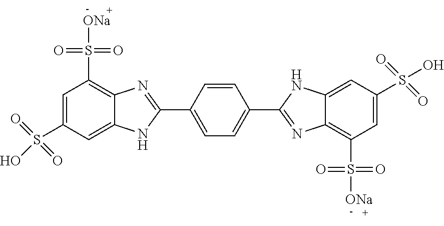

This is commercially available as Neo heliopan AP (from Symrise Shanghai Ltd) or as Sunsafe DPDT (from Uniproma). It is generally used in the water solubilized form after neutralization to deliver desired benefit.

Terephthalylidene dicamphor sulfonic acid (TDSA) has the structure

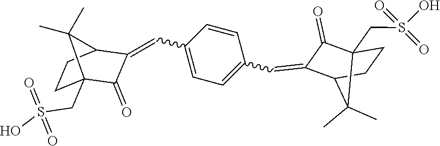 [SMILES: CC1(C)C2CCC1(CS(=O)(=O)O)C(=O)C2=Cc1ccc(C=C2C(=O)C3(CS(=O)(=O)O)CCC2C3(C)C)cc1]

It is also known as Ecamsule. This is commercially available as Mexoryl SX (U.S. Pat. No. 4,585,597) by L′ Oreal or Sunsafe TDSA (from Uniproma). It can be used as parent acid or its salts in water solubilized form to deliver the desired benefit.

The wash-off composition preferably comprises 0.1 to 10 wt %, more preferably 0.25 to 8 wt %, even more preferably 0.5 to 5% water soluble UVA sunscreen. The composition preferably comprises 0.1 to 10 wt %, more preferably 0.25 to 8 wt %, even more preferably 0.5 to 5% water soluble UVB sunscreen. There are certain sunscreens which have both a UVA screening efficacy as well as a UVB screening efficacy. Such sunscreens provide both UVA and UVB protection e.g. benzophenone-4. Benzophenone-4 (Sulisobenzone) has the chemical structure:

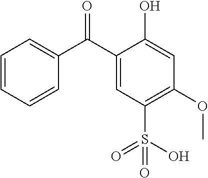 [SMILES: COc1cc(O)c(C(=O)c2ccccc2)cc1S(=O)(=O)O]

Such sunscreens may also be included in the wash-off composition of the invention. When included, it is to be understood that it is taken as having included one type of sunscreen i.e. either a UVA sunscreen or a UVB sunscreen and not both. Thus, when included as a UVA sunscreen, it is essential to include another sunscreen as a UVB sunscreen. Similarly, when included as a UVB sunscreen, it is essential to include another sunscreen as a UVA sunscreen.

The water-soluble sunscreens for inclusion in the wash-off composition of the present invention are generally commercially available in the acid form. When included in the composition in the acid form (pre/partially neutralized forms) i.e. having sulphonic acid group (—SO₃H) the wash-off composition additionally comprises a neutralising agent to convert the acid form into the salt form, in which form it is known to be active as a sunscreen with an exception to Mexoryl SX which can act as sunscreen with and without neutralization. When included, the neutralising agent is preferably in 0.05% to 4% by weight of the composition. The neutralising agent is preferably an inorganic or an organic alkali. Organic alkali is preferably an amine such as triethanol amine or diethanol amine. The present inventors have observed that the inorganic alkali is especially preferred. Preferred are alkali metal hydroxides. Most preferred metal hydroxide for inclusion as neutralising agent in the composition of the invention are sodium hydroxide or potassium hydroxide.

One of the objects of the first step of the method of the invention is to ensure that the sunscreen is deposited in as high an amount as possible on the skin through the wash-off process. Without wishing to be bound by theory, the inventors believe that this is achieved through the select combination of sunscreens chosen by their structure and solubility in the select foaming surfactant composition which determine their partition between rinse away water and cleansing surface resulting in high sunscreen deposition.

The wash-off composition of the invention comprises a mild surfactant which is delivered in the form of a foam which aids in the cleaning action. To enable cleaning, the mild surfactant is included in 3 to 80% preferably 6 to 80%, further more preferably 10 to 75% by weight of the composition. By a mild surfactant is meant that the Zein Number is less than 100 or or has a value of less than 6% zein dissolution weight %. The Zein dissolution wt % is defined in the reference U.S. Pat. No. 9,655,866B2 “Mild antibacterial cleansing compositions”.

The mild surfactant is preferably an anionic surfactant which is selected from one or more of an isethionate, glycinate, cocoate, olefin sulphonate, sulphoacetate and sulphosuccinate surfactant. Preferred mild anionic surfactants are chosen from one or more of sodium cocoyl isethionates, potassium cocoyl glycinate and potassium cocoate, alpha olefin sulphonate (AOS), sodium lauryl sulfoacetate, alkyl ether sulfoacetates, sodium dioctylsulphosuccinate, and alkyl ether sulfosuccinates. These anionic surfactants are preferably present at a level of from 1 to 50%, preferably 2 to 35%, further more preferably from 10 to 35% by weight of the wash-off composition.

The wash-off composition of the invention preferably comprises a mild amphoteric surfactant which may be chosen from one or more of cocoamidopropyl hydroxysultaine, coco amidopropyl betaine, sodium lauroamphoacetate and sodium cocoamphoacetate.

When included the wash-off composition comprises from 0.1 to 10 wt. %, preferably from 0.5 to 8 wt. %, more preferably from 1 to 5 wt. % of the mild amphoteric surfactant.

The wash-off composition of the invention preferably comprises a mild non-ionic surfactant which may be chosen from one or more of Polysorbate 20, Polysorbate 60, sorbitan laurate, decyl glucoside, and lauryl glucoside.

When included the wash-off composition comprises more than 0.5 but less than 30%, preferably from 1 to 20% by weight of the mild non-ionic surfactant.

The wash-off composition of the invention comprises a cosmetically acceptable carrier. The cosmetically acceptable carrier is preferably an aqueous solution/dispersion of cosmetically acceptable adjuvents. Water is generally included in the wash-off composition of the invention. The wash-off composition is generally formulated in liquid, emulsion or gel form where the water content is generally in the range of 50 to 95%, preferably in the range of 60 to 85% by weight of the composition.

Additional adjuvents which may be used to make up the cosmetically acceptable carrier of the wash-off composition are described below.

The wash-off composition preferably comprises a polyhydric alcohol (also called polyol) or mixture of polyols. Polyol is a term used herein to designate a compound having multiple hydroxyl groups (at least two, preferably at least three) which is highly water soluble. Many types of polyols are available including: relatively low molecular weight short chain polyhydroxy compounds such as glycerol and propylene glycol; sugars such as sorbitol, manitol, sucrose and glucose; modified carbohydrates such as hydrolyzed starch, dextrin and maltodextrin, and polymeric synthetic polyols such as polyalkylene glycols, for example polyoxyethylene glycol (PEG) and polyoxypropylene glycol (PPG). Especially preferred polyols are glycerol, sorbitol and their mixtures. Most preferred polyol is glycerol. In a preferred embodiment, the bars of the invention comprise 0 to 8%, preferably 1 to 7.5% by wt. polyol

A preferred wash-off composition may additionally include up to 30 wt % benefit agents. Preferred benefit agents include moisturizers, emollients, and anti-ageing compounds.

Other optional ingredients like anti-oxidants, perfumes, polymers, chelating agents, colourants, deodorants, dyes, enzymes, foam boosters, germicides, anti-microbials, lathering agents, pearlescers, skin conditioners, stabilizers or superfatting agents, may be added in suitable amounts in the process of the invention. Preferably, the ingredients are added after the saponification step. Sodium metabisulphite, ethylene diamine tetra acetic acid (EDTA), borax or ethylene hydroxy diphosphonic acid (EHDP) are preferably added to the formulation.

The present invention includes a second step of the method which comprises applying a leave-on composition comprising a sunscreen and a cosmetically acceptable vehicle on to the desired topical surface e.g a skin surface. By a leave-on composition is meant a composition that is applied on the desired topical surface and left thereon till such time that the surface is cleaned usually after several hours or after one day when a person normally has his bath or shower or any other personal cleansing routine. The leave-on composition of the present invention can be in the form of a liquid, lotion, cream, spray, stick or gel. “Skin” as used herein is meant to include skin on the face and body (e.g., neck, chest, back, arms, underarms, hands, legs, buttocks and scalp) and especially to the sun exposed parts thereof. The leave-on composition of the invention is also of relevance to application on any other keratinous substrates of the human body other than skin e.g. scalp and hair where products may be formulated with specific aim of providing photo-protection.

The sunscreen in the leave-on composition is an organic UVA sunscreen, an organic UVB sunscreen, an inorganic sunblock or mixtures thereof preferably a combination of an organic UVB sunscreen and an organic UVA sunscreen.

Preferably, the leave-on composition comprises a UVA organic sunscreen that absorbs UVA radiations and prevent them from reaching a surface e.g. skin of a user. Examples of sunscreens that may be used as UVA organic sunscreen in the composition are dibenzoylmethane compound, bisdisulizole disodium (commercially available as Neo Heliopan® AP), diethylamino hydroxybenzoyl hexyl benzoate (commercially available as Uvinul® A Plus), Ecamsule (commercially available as Mexoryl SX), Dimethoxyphenyl-[1-(3,4)]-4,4-dimethyl 1,3-pentanedione, and Methyl anthranilate. Preferably, sunscreen that may be used as UVA organic sunscreen in the leave-on composition is selected from a dibenzoylmethane compound or diethylamino hydroxybenzoyl hexyl benzoate, preferably a dibenzoylmethane compound. Examples of sunscreen of dibenzoymethane compound that may be used as UVA organic sunscreen in the leave-on composition include butyl methoxydibenzoylmethane or BMDM (commercially available as Parsol® 1789 or Avobenzone), 2-methyldibenzoylmethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane or 2,6-dimethyl-4-tert-butyl-4′-methoxy-dibenzoylmethane. Most preferably, dibenzoylmethane compound that may be used as UVA organic sunscreen is BMDM.

Preferably, the leave-on composition comprises from 0.01 to 10 wt %, more preferably from 0.1 to 8 wt %, even more preferably from 1 to 6 wt %, further more preferably 1 to 5 wt %, still more preferably from 1 to 3 wt %, yet more preferably from 1 to 2.5 wt % of one or more UVA organic sunscreens.

Preferably, the leave-on composition may comprise a UV-B organic sunscreen. UVB organic sunscreens absorb UVB radiations and prevent them from reaching a surface e.g. skin of a user. Examples of UVB organic sunscreens that may be used in the composition include compounds from the class of cinnamic acid, salicylic acid, diphenyl acrylic acid, Benzylidene camphor and derivatives thereof. Examples of such compounds include 2-ethylhexyl salicylate (commercially available as Octisalate™), 3,3,5-Trimethylcyclohexyl 2-hydroxybenzoate (commercially available as Homosalate™), Ethylhexyl Methoxycinnamate (commercially available as NeoHelipan® AV), 2-ethylhexyl 2-cyano-3,3-diphenylacrylate (OCR; commercially available as Octocrylene™), 2-Hydroxy-4-methoxybenzophenone (commercially available as Oxybenzone™), 2-ethyl-hexyl-4-methoxy cinnamate (MCX; commercially available as Parsol MCX™), 3-Benzylidene camphor, Benzylidene camphor sulfonic acid, DEA methoxycinnamate, Cinoxate, Diethylhexyl butamido triazone, Digalloyl trioleate, Diisopropyl methyl cinnamate, Ethyl dihydroxypropyl PABA, Ethylhexyl dimethoxy benzylidene dioxoimidazoline propionate, Ethylhexyl dimethyl PABA, Ethylhexyl triazone, Ferulic acid, Glyceryl ethylhexanoate dimethoxycinnamate, Glyceryl PABA, Isoamyl p-methoxycinnamate, Isopentyl trimethoxycinnamate trisiloxane, Isopropyl benzyl salicylate, Isopropyl methoxycinnamate, 4-Methylbenzylidene camphor, PABA, PEG-25 PABA, Pentyl dimethyl PABA, Phenyl benzimidazole sulfonic acid, Polyacrylamido methylbenzylidene camphor, Polysilicone-15, Salicylic acid, TEA salicylate and mixtures thereof. Preferably, UVB organic sunscreens that may be used in the leave-on composition are selected from OCR, MCX. phenyl benzimidazole sulphonic acid, ethyl hexyl salicylate and mixtures thereof.

When incorporated in the composition, UVB organic sunscreens may preferably be incorporated from 0.1 to 10 wt %, more preferably from 0.5 to 7 wt %, even more preferably from 1 to 5 wt %, further more preferably from 1 to 3.5 wt %, yet more preferably from 1 to 3 wt %, still more preferably 1 to 2.5 wt % in the leave-on composition.

Preferably, the leave-on composition may comprise a UVB/A sunscreen which are sunscreens that absorb both UVB and UVA radiations and prevent them from reaching the applied surface. Examples of UVB/A sunscreens that may be used in the composition include Benzophenone derivatives. Examples of such compounds are Benzophenone-1, Benzophenone-2, Benzophenone-3, Benzophenone-4, Benzophenone-5, Benzophenone-6, Benzophenone-8, Benzophenone-9, Beta-2-Glucopyranoxy propyl hydroxy benzophenone, Bis-ethylhexyloxyphenol methoxyphenyl triazine (Tinosorb S), Drometrizole, Drometrizole trisiloxane, Methylene bis-benzotriazolyl tetramethylbutylphenol (Tinosorb M).

Preferably, the leave-on composition comprises from 0.01 to 10 wt %, more preferably from 0.1 to 8 wt %, even more preferably from 1 to 6 wt %, further more preferably 1 to 5 wt %, still more preferably from 1 to 3 wt %, yet more preferably from 1 to 2.5 wt % of one or more UVB/A organic sunscreens.

Preferably, the composition may comprise an inorganic sunblock. Examples of inorganic sunblock that may be used in the composition include zinc oxide (ZnO), iron oxide, silica, such as fumed silica and titanium dioxide (TiO₂). Preferably, inorganic sunblock that may be used in the composition are selected from TiO₂, ZnO and mixtures thereof.

When incorporated the composition, inorganic sunblock may preferably be incorporated from 0.1 to 25% wt %, more preferably from 0.5 to 20 wt %, even more preferably from 1 to 15 wt %, further more preferably from 1 to 10 wt %, yet more preferably from 1 to 5 wt %, still more preferably 1 to 3 wt %, in the composition.

The leave-on composition may comprise one or more agents for providing glow to the skin. Examples of the agents include niacinamide, picolinamide, 4-alkyl substituted resorcinol, vitamin B6, vitamin C, vitamin A, glutathione precursors, galardin, adapalene, aloe extract, ammonium lactate, arbutin, azelaic acid, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, gluco pyranosyl-1-ascorbate, gluconic acid, glycolic acid, green tea extract, 4-Hydroxy-5-methyl-3 [2H]-furanone, 4-hydroxyanisole and its derivatives, 4-hydroxybenzoic acid derivatives, hydroxycaprylic acid, inositol ascorbate, lactic acid, lemon extract, linoleic acid, magnesium ascorbyl phosphate, 5-octanoyl salicylic acid, salicylic acid, 3,4,5-trihydroxybenzyl derivatives, acetylglucosamine, pitera extract, symwhite, calcium pantothenate (Melano-block), seppiwhite, soybean extract (bowman birk inhibitor), 12-hydroxystearic acid and mixtures thereof. When used in the composition, 12-hydroxystearic acid is used as a skin glow agent and not as a fatty acid.

Preferably, the agent for providing glow to the skin is selected from the group consisting of niacinamide, picolinamide, isonicotinamide, 12-hydroxystearic acid, 4-alkyl substituted resorcinol and mixtures thereof. Preferably the skin glow agent used in the leave-on composition is niacinamide. More preferably, the skin glow agent selected is 4-alkyl substituted resorcinol selected from the group consisting of 4-ethyl resorcinol (4-ER), 4-isopropyl resorcinol (4-IPR), 4-butyl resorcinol (4-BR), 4-pentyl resorcinol (4-PR), 4-phenylethyl resorcinol (4-PER), 4-HR and mixtures thereof. Even more preferably, the agent is selected from the group consisting of 4-ER, 4-HR and mixtures thereof. Further more preferably, the agent selected is 4-HR.

Preferably, the leave-on composition comprises from 0.001 to 10 wt %, more preferably from 0.01 to 8 wt %, even more preferably from 0.1 to 7 wt %, further more preferably from 0.1 to 5 wt %, still more preferably from 0.1 to 3 wt %, yet more preferably from 0.1 to 1 wt % and still further more preferably from 0.25 to 0.5 wt % of a skin glow agent.

The leave-on composition comprises a cosmetically acceptable vehicle. The cosmetically acceptable vehicle is preferably in the form of an oil, liquid, stick, cream, lotion, spray or gel. The cosmetically acceptable vehicle preferably includes ingredients like surfactant e.g. non-ionic surfactant, fatty acid, soap, water, polymer, emollients, solvents, powders, preservatives, or optional ingredients. Details on the various possible ingredients for inclusion in the cosmetically acceptable vehicle are given below.

Preferably, the leave-on composition comprises a nonionic surfactant. More preferably the nonionic surfactant is selected from those having HLB value in the range 9 to 20, preferably 10 to 19, more preferably 12 to 18, even more preferably 13 to 17 and yet more preferably 15 to 17.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various surfactants are given below:
A value <10: Lipid soluble (water insoluble)
A value >10: Water soluble
A value from 4 to 8 indicates an anti-foaming agent
A value from 7 to 11 indicates a W/O (water in oil) emulsifier
A value from 12 to 16 indicates oil in water emulsifier
A value from 11 to 14 indicates a wetting agent
A value from 12 to 15 is typical of detergents
A value of 16 to 20 indicates a solubiliser or a hydrotrope.

Preferably, the nonionic surfactant having HLB value in the range 9 to 20 is selected from fatty alcohol ethoxylates, alkyl phenol ethoxylates, polyoxyethylene sorbitan alkyl esters and mixtures thereof. Preferably, the nonionic surfactants are ones with at least 9 alkylene oxide groups preferably at least 9 ethylene oxide groups.

Examples of fatty alcohol ethoxylates that may be used as nonionic surfactants in the composition include polyoxyethylene lauryl ether (HLB=16.9; commercially available as Brij® 35), polyoxyethylene (20) cetyl ether (HLB=16; commercially available as Brij® 58), polyethylene glycol octadecyl ether (HLB=18.8; commercially available as Brij® 700) and Laureth-9 (C12EO9; HLB=14.3; commercially available as Brij® L9).

Examples of alkyl phenol ethoxylates that may be used as nonionic surfactant in the composition include octylphenol ethoxylate (HLB=15.5; commercially available as Triton™ X165), octylphenol ethoxylate (HLB=17.6; commercially available as Triton™ X405) and octylphenol ethoxylate (HLB=18.4; commercially available as Triton™ X705).

Examples of polyoxyethylene sorbitan alkyl esters that may be used as the nonionic surfactant in the composition include polyoxyethylenesorbitan monolaurate (HLB=13.3; commercially available as Tween® 21), polyoxyethylenesorbitan monolaurate (HLB=16.7; commercially available as Tween® 20), Polyoxyethylenesorbitan monopalmitate (HLB=15.6; commercially available as Tween® 40) and polyoxyethylene sorbitan monostearate (HLB=14.9; commercially available as Tween® 60).

Even more preferably, the nonionic surfactant having HLB value in the range 9 to 20 that may be present in the composition is fatty alcohol ethoxylate with saturated carbon chain having HLB higher than 15.5.

Preferably, the leave-on composition comprises 0.5 to 5 wt %, more preferably 1 to 4 wt %, even more preferably from 2 to 3 wt % nonionic surfactant having HLB in the range 9 to 20.

Preferably, the leave-on composition comprises fatty acid. Fatty acids when present in a composition along with a soap provides the so-called vanishing cream effect, i.e. a composition, when applied on to the human skin, vanishes on the skin leaving behind no significant streaks of the composition. Preferably, the leave-on composition comprises fatty acids having 10 to 30, more preferably 12 to 25, even more preferably 14 to 20, further more preferably 16 to 18 carbon atoms. Examples of fatty acids that may be used in the composition include pelargonic, lauric, myristic, palmitic, stearic, isostearic, oleic, linoleic, arachidic, behenic, erucic acid and mixtures thereof. Preferably, the fatty acid that may be used is stearic acid or palmitic acid or a mixture thereof. The fatty acid in the present invention is preferably hystric acid which is substantially (generally about 90 to 95%) a mixture of stearic acid and palmitic acid in a ratio of between 55:45 to 45:55.

Preferably, the leave-on composition comprises from 2.25 to 25 wt %, more preferably from 4 to 22 wt %, even more preferably from 6 to 20 wt %, further more preferably from 8 to 19 wt % and still more preferably from 10 to 18 wt % and yet more preferably from 12 to 16 wt % fatty acid.

Preferably, the leave-on composition comprises soap. Soap, when present in combination with fatty acid in the composition, provides the vanishing cream effect. Preferably, soap in the leave-on composition is generally prepared by in-situ neutralization of fatty acid that may be present in the composition. Thus, it is preferred that the soap has a carbon chain length that corresponds to the chain length of fatty acid in the composition. The soap is formed from the fatty acid through use of alkali metal hydroxides e.g. sodium hydroxide or potassium hydroxide. Of the two, potassium hydroxide is more preferred. Thus, the soap is preferably a potassium soap (potassium salt of fatty acid).

Preferably, the leave-on composition comprises from 0.1 to 10 wt %, more preferably from 0.25% to 8 wt %, even more preferably from 0.5 to 7 wt %, further more preferably from 0.5 to 5 wt % soap, even further more preferably 0.5% to 3%.

Preferably, the leave-on composition comprises water in amount from 5 to 99.9 wt %, more preferably from 10 to 95 wt %, even more preferably from 15 to 90 wt %, further more preferably from 20 to 80 wt %, still more preferably 25 to 75 wt % and yet more preferably 30 to 70 wt %.

Preferably, the leave-on composition comprises a polymer. The polymer acts as thickener in the composition and improves sensorial properties of the composition.

The polymer is preferably selected from the following classes:
acrylate/R-methacrylate copolymer e.g. acrylates/steareth-20 methacrylate copolymer (commercially available as Aculyn™ 22) and acrylates/beheneth-25 methacrylate copolymer (commercially available as Aculyn™ 28), acrylate/R-methacrylate crosspolymer e.g. acrylates/steareth-20 methacrylate crosspolymer (commercially available as Aculyn™ 88),
acrylates copolymer (commercially available as Aculyn™ 33),
acrylate/R-alkyl acrylate crosspolymer e.g. acrylates/C10-C30 alkyl acrylate crosspolymer (commercially available as Pemulen™ TR-2),
copolymer of ammonium acryloyldimethyltaurate with vinyl pyrrolidone (commercially available as Aristoflex® AVC),
copolymer of sodium acryloyldimethyltaurate with vinyl pyrrolidone (commercially available as Aristoflex® AVS); and
crosspolymer of acryloyldimethyltaurate with R-alkyl acrylate and methyacrylate e.g. Ammonium acryloyldimethyltaurate/beheneth-25 methacrylate crosspolymer (commercially available as Aristoflex® HMB and Aristoflex® BLV).

Preferably, the leave-on composition comprises 0.1 to 5 wt %, more preferably 0.25 to 4.5 wt %, even more preferably 0.5 to 4 wt %, further more preferably from 0.75 to 3.5 wt %, still more preferably from 0.75 to 2.75 wt % polymer.

Preferably, the leave-on composition comprises emollients. Examples of emollients that may be used in the leave-on composition include stearyl alcohol, glyceryl monoricinoleate, mink oil, cetyl alcohol, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

Preferably, the leave-on composition comprises solvents. Examples of solvents that may be used in the composition include ethyl alcohol, isopropanol, acetone, ethylene glycol ono ethyl ether, diethylene glycol mono butyl ether, diethylene glycol mono ethyl ether and mixtures thereof.

Preferably, the leave-on composition comprises powders. Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Preferably, the leave-on composition comprises preservatives to protect against the growth of potentially harmful microorganisms. Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. The alkane diols that are suitable for use as preservative are C₆-C₁₂ alkanes that are vicinally substituted with hydroxy groups. Illustrative examples include 1,2-octane diol (caprylyl glycol), 2,3-octane diol, 1,2-nonane diol, 1,2-decane diol, 1,2-hexane diol, 3,4-octane diol, mixtures thereof or the like where caprylyl glycol is typically the most preferred.

When present in the leave-on composition, preservatives are added preferably in an amount 0.001 to 5 wt %, more preferably 0.01 to 3 wt % and most preferably 0.02 to 2 wt %, even most preferably 0.25 to 1.5%.

Preferably, the leave-on composition comprises a range of other optional ingredients that include antioxidants, binders, buffering agents, colorants, astringents, fragrance, opacifying agents, conditioners, exfoliating agents, pH adjusters, skin sensates, skin soothing agents, and skin healing agents.

The present invention relates to the method as described in detail herein above viz, the step of washing the surface with the wash-off composition followed by rinsing followed by applying a leave-on composition. It is preferrable to dry the skin after the rinsing step by dabbing the surface with a towel to leave the surface substantially dry to touch. The step of applying the leave-on composition after the rinsing off step may be carried out anywhere from a minute to an hour after the rinsing step. Preferably the step of applying the leave-on composition is from one minute to 30 minutes, more preferably one minute to 10 minutes, further more preferably from one minute to five minutes after the rinsing step. Preferably, the method is non-therapeutic or cosmetic in nature.

The invention also relates to a method of providing an SPF of 10, at least 20, preferably at least 30, more preferably at least 50, to the topical surface of the body. Preferably, the method is non-therapeutic or cosmetic in nature. The invention also relates to a method of providing an UVAPF of 4 to skin, of at least 8, preferably at least 12, more preferably at least 16 to the topical surface of the body. Preferably, the method is non-therapeutic or cosmetic in nature.

According to another aspect of the present invention, there is provided a kit comprising a hand held container comprising a pump capable of generating a foam, said container comprising a wash-off composition comprising 0.1 to 10 wt % of a water-soluble UVA sunscreen; 0.1 to 10 wt % of a water-soluble UVB sunscreen; 3 to 80 wt %, preferably 6 to 80 wt % a mild surfactant selected from one or more of an anionic, zwitterionic or non-ionic surfactant; and a cosmetically acceptable carrier; a leave-on composition comprising a sunscreen and a cosmetically acceptable vehicle; and instructions for use. Preferably, the use is non-therapeutic or cosmetic in nature.

The invention will now be demonstrated with the help of the following non-limiting examples.

### Examples

### Wash off Compositions 1-2:

The following skin cleansing compositions were prepared as shown in Table-1 below:

TABLE 1

| | 1 | 2 |
|---|---|---|
| Ingredients | wt % | wt % |
| Potassium Cocoyl Glycinate 19% + | 41.1 | 41.1 |
| Potassium Cocoate 11% + Water | | |
| Sodium Lauroamphoacetate 25% | 12.8 | 12.8 |
| Propylene Glycol Pharmacopeia Grade | 6.0 | 6.0 |
| Glycerin Pharmacopeia Grade 99.5% | 6.0 | 6.0 |
| Polysorbate 20 HPC | 0.3 | 0.3 |
| Citric Acid Monohydrate | 0.18 | 0.18 |
| Phenoxyethanol | 0.6 | 0.6 |
| Potassium Hydroxide 85% | 0.002 | 0.002 |
| Niacinamide Pharmacopoeia Grade | 0.001 | 0.001 |
| (crystalline/fine powder) | | |
| Fragrance | 0.85 | 0.45 |
| Stay C 50 | 0.01 | 0.05 |
| Sunscreen (1% Ensulizole, 0.3% | 1.30 | 1.30 |
| Neoheliopan AP) preneutralized | | |
| Water | to 100 | to 100 |

Leave-on Compositions: 3-5: The following leave-on compositions were prepared as shown in Table-2 below:

TABLE 2

| | 3 | 4 | 5 |
|---|---|---|---|
| Ingredients | Wt % | Wt % | Wt % |
| Fatty Acid C16:C18 55:45 | 17.00 | 6.00 | 0.00 |
| Cetyl Alcohol Solid High C16 | 0.53 | 0.37 | 0.00 |
| Carbomer | 0.00 | 0.40 | 0.00 |
| Glyceryl Stearate (50% monostearate, low | 0.00 | 0.65 | 0.00 |
| glycerol) | | | |
| Glycol Stearate 97% + Stearamide AMP 3% | 0.00 | 1.39 | 0.00 |
| Peg-100 Stearate (Vegetable or Synthetic) | 0.00 | 1.19 | 0.00 |
| Dimethicone 350 cst | 0.50 | 0.00 | 0.00 |
| Iso Propyl Myristate | 0.75 | 0.00 | 0.00 |
| Ethylhexyl Methoxycinnamate | 0.75 | 4.00 | 7.50 |
| Butyl Methoxydibenzoylmethane | 0.40 | 1.00 | 0.00 |
| Titanium Dioxide 74% + Aluminum | 0.60 | 0.00 | 0.00 |
| Hydroxide + Stearic Acid -Rutile | | | |
| Titanium Dioxide 99.4% + Alumina + | 0.30 | 0.00 | 0.00 |
| Zinc Oxide-Rutile | | | |
| Ethylhexyl Salicylate | 0.00 | 1.00 | 0.00 |
| Dimethicone 50 cst | 0.00 | 0.40 | 0.00 |
| Phenoxyethanol | 0.20 | 0.00 | 0.00 |
| Niacinamide Pharmacopoeia Grade | 1.25 | 0.10 | 1.25 |
| (crystalline/fine powder) | | | |
| Allantoin | 0.20 | 0.00 | 0.10 |
| Octocrylene | 0.00 | 1.00 | 0.00 |
| Caprylyl Glycol | 0.00 | 0.50 | 0.00 |
| Cyclopentasiloxane85% + | 0.00 | 0.00 | 20.00 |
| DimethCrosspoly12% | | | |
| Potassium Chloride | 0.00 | 0.00 | 2.00 |
| PEG-10 Dimethicone | 0.00 | 0.00 | 1.00 |
| Titanium dioxide 93% + Aluminum | 0.00 | 0.00 | 0.30 |
| hydroxide + Dimethicone-Rutile | | | |
| Zinc Oxide + Cyclopentasiloxane | 0.00 | 0.00 | 8.40 |
| Capric/Caprylic Triglyceride 60:40 | 0.00 | 0.00 | 1.50 |
| Glycerin Refined Grade 98% | 1.00 | 2.50 | 8.00 |
| Sodium Hydroxide flake 97% | 0.00 | 0.23 | 0.58 |
| Phenylbenzimidazole Sulfonic Acid | 0.00 | 1.50 | 4.00 |
| Potassium hydroxide | 0.56 | 0.54 | 0.00 |
| Xanthan Gum (Cosmetic Grade) | 0.00 | 0.30 | 0.00 |
| Water | To 100 | To 100 | To 100 |

Using the above samples, the following experiments were carried out

For wash-off composition the protocol used was as follows: A pre-wet vitro skin was applied with diluted wash-off product of Composition 1 or 2. The vitro skin was rinsed off immediately post product application and kept for drying in dark. After the drying step, vitro skin was exposed to UV light and transmittance scan was recorded. This scan gives the transmittance as a function of wavelength (290-400 nm) for a given sample. For a single vitro skin sample four different spots were scanned. The same was repeated for 2 vitro skin samples. The data reported is thus an average over 8 readings. The reference transmittance scan was obtained using blank vitro skin, with glycerine spread on it as control. The transmittance values were used to arrive at the SPF and UVA PF values using the software provided with the instrument.

For leave-on composition the protocol used was as follows: The vitro skin post washing with Composition 1 or 2 was applied with 2 mg/cm² of leave-on product (Composition 3 or 4 or 5) on the same side of the vitro skin where cleansing was performed to mimic the consumer scenario. The vitro skin applied was then allowed to dry in the dark for 20-30 minutes to simulate recommended drying time from in vivo ISO 24444 methods. After drying, vitro skin was exposed to UV light and transmittance scan was recorded. This scan gives the transmittance as a function of wavelength (290-400 nm) for a given sample. For a single vitro skin sample four different spots were scanned. The same was repeated for 2 vitro skin samples. The data reported is thus an average over 8 readings. The reference transmittance scan was obtained using blank vitro skin, with glycerine spread on it as control. The transmittance values were used to arrive at the SPF and UVA PF values using the software provided with the instrument.
Example—1: The SPF and UVAPF of the substrate was measured after washing it with wash-off composition 1
Example—2: The SPF and UVAPF of the substrate was measured after washing it with wash-off composition 2
Example—3: The SPF and UVAPF of the substrate was measured after applying leave-on composition 3.
Example—4: The SPF and UVAPF of the substrate was measured after applying leave-on composition 4.
Example—5: The SPF and UVAPF of the substrate was measured after applying leave-on composition 5.
Example—6: The SPF and UVAPF of the substrate was measured after washing substrate with composition 1 followed by applying leave-on composition 3.
Example—7: The SPF and UVAPF of the substrate was measured after washing substrate with composition 1 followed by applying leave-on composition 4.
Example—8: The SPF and UVAPF of the substrate was measured after washing substrate with composition 1 followed by applying leave-on composition 5.
Example—9: The SPF and UVAPF of the substrate was measured after washing substrate with composition 2 followed by applying leave-on composition 3.
Example—10: The SPF and UVAPF of the substrate was measured after washing substrate with composition 2 followed by applying leave-on composition 4.
Example—11: The SPF and UVAPF of the substrate was measured after washing substrate with composition 2 followed by applying leave-on composition 5.

The SPF and the UVA PF of the various Examples as listed above were measured and the data is summarised in Table-3 below:

TABLE 3

| Example | SPF | UVAPF |
|---|---|---|
| 1 | 22.1 | 2.9 |
| 2 | 23.5 | 3.0 |
| 3 | 7.7 | 4.6 |
| 4 | 53.4 | 7.9 |
| 5 | 66.0 | 6.5 |
| 6 | 102.8 | 13.4 |
| 7 | 94.6 | 13.4 |
| 8 | 98.1 | 12.8 |
| 9 | 77.1 | 9.8 |
| 10 | 128.0 | 19.9 |
| 11 | 106.6 | 13.0 |

The data in the table-3 above indicates that the method as per the invention (Examples 6-11) provides for synergistic improvement in SPF and UVAPF over the individual steps of the method (Examples 1 to 5).

1. A method of providing high SPF to a topical surface of a body comprising the steps of:
(a) washing the surface with a foam wash-off composition comprising;
(i) 0.1 to 10 wt % of a water-soluble UVA sunscreen;
(ii) 0.1 to 10 wt % of a water-soluble UVB sunscreen;
(iii) 3 to 80 wt %, of a mild surfactant selected from one or more of an anionic, zwitterionic and non-ionic surfactant; and
(iv) a cosmetically acceptable carrier;
(b) rinsing the surface with water; followed by
(c) applying on the surface a leave-on composition comprising a sunscreen and a cosmetically acceptable vehicle.

2. The method as claimed in claim 1, wherein said anionic surfactant is selected from one or more of an isethionate, glycinate, cocoate, olefin sulphonate, sulphoacetate, and sulphosuccinate surfactant.

3. The method as claimed in claim 1, wherein said zwitterionic surfactant is selected from one or more of a sultaine, betaine, and amphoacetate surfactant.

4. The method as claimed in claim 1, wherein said non-ionic surfactant is selected from one or more of a polysorbate, sorbitan, polyethylene glycol, and glucoside surfactant.

5. The method as claimed in claim 1, wherein solubility in water of said water-soluble UVA sunscreen and said water-soluble UVB sunscreen is higher than 10 g/l at 25 °C.

6. The method as claimed in claim 1, wherein the water soluble UVB sunscreen is selected from phenyl benzimidazole sulfonic acid, benzylidene camphor sulfonic acid, or mixtures thereof.

7. The method as claimed in claim 1, wherein said water soluble UVA sunscreen is selected from disodium phenyl dibenzimidazole tetra sulfonate, terephthalylidene dicamphor sulfonic acid, or mixtures thereof.

8. The method as claimed in claim 1, wherein the sunscreen in the leave-on composition is an organic UVA sunscreen, an organic UVB sunscreen, an inorganic sunblock, or mixtures thereof.

9. The method as claimed in claim 8, wherein said organic UVA sunscreen is butyl methoxydibenzoylmethane or diethylamino hydroxybenzoyl hexyl benzoate; said organic UVB sunscreen is ethylhexyl methoxycinnamate, Octocrylene, phenyl benzimidazole sulphonic acid, or ethyl hexyl salicylate; and said inorganic sunblock is zinc oxide or titanium dioxide.

10. The method as claimed in claim 1, wherein said cosmetically acceptable vehicle of the leave-on composition is in the form of a cream, stick, lotion, gel, or spray.

11. The method as claimed in claim 1, wherein the step of applying the leave-on composition is carried out one minute to an hour after the step of rinsing said surface with water.

12. The method as claimed in claim 1 further comprising obtaining an SPF of at least 10.

13. A kit comprising
(a) a hand held container comprising a pump capable of generating a foam, said container comprising a wash-off composition comprising:
0.1 to 10 wt % of a water-soluble UVA sunscreen;
0.1 to 10 wt % of a water-soluble UVB sunscreen;
3 to 80 wt %, preferably 6 to 80 wt % a mild surfactant selected from one or more of an anionic, zwitterionic or non-ionic surfactant; and
a cosmetically acceptable carrier;
(b) a leave-on composition comprising a sunscreen and a cosmetically acceptable vehicle; and
(c) Instructions for use.

14. The method of claim 1, wherein the foam wash-off composition comprises from 0.1 to 1 wt % of phenyl benzimidazole sulfonic acid from 0.1 to 0.3 wt % and disodium phenyl dibenzimidazole tetra sulfonate.

15. The method of claim 2, wherein the foam wash-off composition comprises from 35 to 50 wt % of the anionic surfactant comprising potassium cocoyl glycinate and potassium cocoate.

16. The method of claim 1, wherein the foam wash-off composition further comprises one or more amphoteric surfactants.

17. The method of claim 16, wherein the foam wash-off composition comprises from 1 to 12.8 wt % of sodium lauroamphoacetate.

18. The method of claim 8, wherein the leave-on composition comprises the organic UVA sunscreen and the organic UVB sunscreen.

19. The method of claim 8, wherein the leave-on composition comprises from 0.1 to 10 wt % of ethylhexyl methoxycinnamate.

20. The method of claim 8, wherein the leave-on composition is free of the inorganic sunblock.
